SEQUENCE LISTING (5 sequences)

SEQ ID NO: 1 - protein, 5 aa, Homo sapiens
VLQRY

SEQ ID NO: 2 - protein, 8 aa, Homo sapiens
TKVNFYAW

SEQ ID NO: 3 - protein, 9 aa, Homo sapiens
SGIRSLTTL

SEQ ID NO: 4 - protein, 6 aa, Homo sapiens
SNFLRG

SEQ ID NO: 5 - protein, 165 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD

(19) United States
(12) Patent Application Publication
(10) Pub. No.: US 2009/0258821 A1
(43) Pub. Date: Oct. 15, 2009

(54) Tissue protective cytokines for the protection, restoration, and enhancement of responsive cells, tissues and organs with an extended therapeutic window

(75) Inventors: Anthony Cerami, Somers, NY (US); Michael Brines, Woodbridge, CT (US); Thomas Coleman, Mt. Kisco, NY (US)

Correspondence Address: JONES DAY, 222 EAST 41ST ST, NEW YORK, NY, 10017, US

(73) Assignee: The Kenneth S. Warren Institute, In

(21) Appl. No.: 11/283,024

(22) Filed: Nov. 18, 2005

Related U.S. Application Data
(63) Continuation of application No. PCT/US04/15863, filed on May 20, 2004.
Application No. PCT/US04/15863 is a continuation-in-part of application No. PCT/US04/15733, filed on May 19, 2004.
(60) Provisional application No. 60/471,661, filed on May 19, 2003.

(51) Int. Cl. A61K 38/19; A61P 43/00

(52) U.S. Cl. 514/8

(57) ABSTRACT

Methods and uses are provided for a pharmaceutical composition with an erythropoietin or a tissue protective cytokine for protecting or restoring function to a responsive cell, tissue, organ or body part function or viability in mammals when administered outside of the therapeutic window of previously approved therapeutics.

### CROSS-REFERENCE TO RELATED APPLICATIONS

A continuation, under 35 U.S.C. § 111(a), of PCT Patent Application Serial No. PCT/US04/15863, filed May 20, 2004, which is incorporated herein by reference in its entirety. A continuation-in-part, under 35 U.S.C. § 111(a), of PCT Patent Application Serial No. PCT/US04/15733, filed May 19, 2004, which is incorporated herein by reference in its entirety, and claims priority under 35 U.S.C. § 119(e)(1) to U.S. provisional application Ser. No. 60/471,661, filed May 19, 2003, which is incorporated herein by reference in its entirety.

### BACKGROUND OF THE INVENTION

Important to the survival of an individual is the body's response to injury, including but not limited to trauma, hypoxia-ischemia, seizure, infection, and poisoning. The human body has developed a two-fold response to injury. Initially, cells directly affected by the injury or trauma die through necrosis, the uncontrolled lysis of the cells. Various cell factors released from the necrotic cells trigger the secondary response to the injury. This response attempts to stop the spread of tissue damage from the localized, primary necrotic site of insult or injury following a catastrophic event such as a stroke, heart attack, or spinal cord trauma, by destroying a much larger mass of otherwise healthy tissue surrounding the injury.

This secondary mechanism evolved as a way of protecting an individual from succumbing to secondary damage from the primary event by reducing the risk of infection and protecting the surrounding tissue from additional injury. The mechanism is initiated by first constraining the blood flow to the damaged area to minimize blood loss, maintain blood pressure and reduce oxygen and nutrient flow to the effected tissue. Within hours of the primary event, certain tissue factors, including tissue necrosis factor (TNF), are released by the damaged cells into their surroundings. These factors attract macrophages, white blood cells specialized in cleaning up damaged tissues. As a result, potentially viable cells within the affected region begin to die, predominantly via programmed cell death, or apoptosis. In apoptosis, a genetic program leads to degradation of nuclear DNA, followed by shrinking and fragmentation of the nucleus. Moderate inflammatory reactions ensue, helping to clean up the damaged tissue and resulting debris from the apoptotic cells. As the inflammation abates, repair mechanisms become activated and ultimately lead to the formation of a scar. The area surrounding the site of primary injury in which these secondary mechanisms take place is referred to as the penumbra.

In light of today's modern therapeutics and overall standard of care, this secondary, body-induced damage to cells and tissues serves no survival function and may lead to further impairment and delays in recovering from the primary injury. Myocardial infarction (heart attack), chronic heart failure, age-related macular degeneration, diabetic retinopathy, diabetic neuropathy, Alzheimer's disease, multiple sclerosis, and Lou Gehrig's disease are all disorders in which apoptosis appears to be an important pathogenic factor. It has been shown in numerous animal injury and disease models involving apoptosis that the pharmacological reduction of apoptosis in the penumbra results in improved functional and histomorphometric outcomes. Each step that induces apoptosis within the penumbra offers a potential target for therapeutic intervention.

Conversely, this means that treatment needs to be rendered before the body has committed itself to that particular step in inducing apoptosis—the therapeutic window. For example, a current treatment for stroke includes the administration of recombinant tissue plasminogen activator (rt-PA), a thrombolytic agent that has been shown to be effective in dissolving clots to restore blood flow to injured areas of the brain. However, it has a therapeutic window of three (3) hours after the onset of a stroke and has only been proven effective when used within this time period. Unfortunately, patients afflicted with a stroke often wait one to two days before seeking medical attention. This is due in part to the public's ignorance of stroke and stroke symptoms, the patient's confusion during the early period of the stroke, and the patient's denial of the illness. Furthermore, depending upon the severity of the stroke and the capacity of the individual following the stroke, it may not be possible for the individual to seek medical attention following the stroke.

Spinal cord injury provides another example of a situation in which the therapeutic window of currently approved therapeutics may be insufficient. Previously, the steroid drug methylprednisone was thought to improve recovery from spinal cord injury when administered within eight (8) hours of the injury, however, recent evidence calls into question the therapeutic value of this treatment. See Hugenholtz, Herman, Methylprednisolone for acute spinal cord injury: not a standard of care, CMAJ, 2003; 168 (9). Once again, individuals prone to these spinal cord injuries may not be able to receive this questionable treatment within the therapeutic window. For example athletes, especially extreme sports enthusiasts, military personnel, construction workers, etc. may receive their injuries in locations or under conditions that would prevent them from receiving treatment within the therapeutic window. Lastly, thrombolytics such as rt-PA (TNKase, Genentech, South San Francisco, Calif. and RETAVASE, Centocor, Inc., Malvern, Pa.) and streptokinase (STREPASE, AstraZeneca LP, Wilmington, Del.) are used to address myocardial infarction (MI), more commonly known as heart attacks. The therapeutic window for many of these thrombolytics is up to 6 hours following the infarct. See Goldberg et al., Impact of Time to Treatment with Tissue Plasminogen Activator on Morbidity and Mortality Following Acute Myocardial Infarction, Am J Cardiol 1998; 82:259-264. Again, this therapeutic window is too short for many individuals afflicted to receive treatment. See Dracup et al., An International Perspective on the Time to Treatment for Acute Myocardial Infarction, Journal of Nursing Scholarship, 2003; 35:4, 317-323.

Thus, a need exists for tissue protective compounds possessing an extended therapeutic window, i.e. a therapeutic window that extends beyond that of currently approved therapeutics within a particular indication—e.g. beyond the three (3) hour therapeutic window for rt-PA in stroke or beyond the eight (8) hour window for methylprednisone for spinal cord injury. In addition, a need exists for a means of pre-treating individuals prone to these injuries so as to provide them with a degree of protection or ameliorate the extent of the damage resulting from the injury.

### BRIEF SUMMARY OF THE INVENTION

The present invention relates to the use of an erythropoietin or a tissue protective cytokine for the preparation of a pharmaceutical composition protection against an injury the restoration of function following the injury in responsive mammalian cells, tissue or organ wherein the pharmaceutical composition is administered at a time outside of a therapeutic window recognized for a currently approved therapeutic for the injury. Preferably, the pharmaceutical composition is administered prior to a therapeutic window recognized for a currently approved therapeutic for the injury, or after the therapeutic window recognized for the currently approved therapeutic for the injury. Additionally, the pharmaceutical composition may be administered to mammal at least at any two of the following three times: (1) prior to a therapeutic window recognized for a currently approved therapeutic for the injury, (2) within the therapeutic window recognized for a currently approved therapeutic for the injury, or (3) after the therapeutic window recognized for the currently approved therapeutic for the injury. Preferably, the pharmaceutical composition is administered a time after the therapeutic window recognized for currently approved therapeutics for the injury and at least one of the following times: (1) prior to a therapeutic window recognized for a currently approved therapeutic for the injury, or (2) within the therapeutic window recognized for a currently approved therapeutic for the injury. Alternatively, the pharmaceutical composition may be administered at all three of the above noted times.

In one embodiment of the invention, the erythropoietin used in the pharmaceutical composition is selected from the group consisting of a chemically modified erythropoietin and a recombinant erythropoietin.

In another embodiment, the pharmaceutical composition includes a tissue protective cytokine that lacks at least one activity selected from the group consisting of increasing hematocrit, vasoconstriction, hyperactivating platelets, pro-coagulant activity and increasing production of thrombocytes. Preferably, the tissue protective cytokine is selected from the group consisting of a chemically modified erythropoietin and a recombinant erythropoietin. Chemically modified erythropoietines suitable as tissue protective cytokines may be one of the following: (I) an erythropoietin having at least no sialic acid moieties; (ii) an erythropoietin having at least no N-linked or no O-linked carbohydrates; (iii) an erythropoietin having at least a reduced carbohydrate content by virtue of treatment of native erythropoietin with at least one glycosidase; (iv) an erythropoietin having at least one or more oxidized carbohydrates; (v) a chemically reduced erythropoietin having at least one or more oxidized carbohydrates; (vi) an erythropoietin having at least one or more modified arginine residues; (vii) an erythropoietin having at least one or more modified lysine residues; (viii) an erythropoietin having at least one modification of the N-terminal amino group of the erythropoietin molecule; (ix) an erythropoietin having at least a modified tyrosine residue; (x) an erythropoietin having at least a modified aspartic acid or glutamic acid residue; (xi) an erythropoietin having at a modified tryptophan residue; (xii) an erythropoietin having at least one amino acid removed; (xiii) an erythropoietin having at least one opening of at least one of the cystine linkages in the erythropoietin molecule; and (xiv) a truncated erythropoietin. In one embodiment, the tissue protective cytokine is an asialo erythropoietin, preferably human asialo erythropoietin. In another embodiment, the tissue protective cytokine is an erythropoietin having at least one carbamoylated lysine residue.

In yet another embodiment of the present invention, the pharmaceutical composition uses a tissue protective cytokine that is a recombinant erythropoietin which is an erythropoietin mutein having one or more altered amino acid residue between position 11 to 15 of SEQ ID NO:5 [SEQ ID NO:1], position 44 to 51 of SEQ ID NO 5 [SEQ ID NO:2], position 100-108 of SEQ ID NO 5[SEQ ID NO:3], or position 146-151 of SEQ ID NO 5 [SEQ ID NO:4].

The pharmaceutical compositions mentioned above may be used to treat such mammalian cells as neuronal, retinal, muscle, heart, lung, liver, kidney, small intestine, adrenal cortex, adrenal medulla, capillary, endothelial, testes, ovary, endometrial, or stem cells. Specifically, the mammalian cells may further be photoreceptor, ganglion, bipolar, horizontal, amacrine, Müller, myocardium, pace maker, sinoatrial node, sinus node, atrioventricular node, bundle of His, hepatocyte, stellate, Kupffer, mesangial, goblet, intestinal gland, enteral endocrine, glomerulosa, fasciculate, reticularis, chromaffin, pericyte, Leydig, Sertoli, sperm, Graffian follicles, primordial follicles, endometrial stroma, and endometrial cells.

Moreover, the above mentioned pharmaceutical composition may be used to provide protection against or restore function following injury related to a seizure disorder, multiple sclerosis, stroke, hypotension, cardiac arrest, ischemia, myocardial infarction, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis, alcoholism, mood disorder, anxiety disorder, attention deficit disorder, autism, Creutzfeldt-Jakob disease, brain or spinal cord trauma or ischemia, heart-lung bypass, chronic heart failure, macular degeneration, diabetic neuropathy, diabetic retinopathy, glaucoma, retinal ischemia, or retinal trauma.

In one embodiment, the pharmaceutical compositions of the present use are capable of therapeutic effect when administered to a mammal at about 8 hours to about 168 hours following the injury. Preferably, the pharmaceutical compositions are capable of therapeutic effect when administered at about 12 hours to about 72 hours following the injury. In another embodiment, the pharmaceutical compositions of the present use are capable of therapeutic effect when administered to a mammal at about 1 to 24 hours prior to the injury. Preferably, the pharmaceutical compounds are capable of therapeutic effect when administered to a mammal at about 6 to 18 hours prior to the injury.

Examples of the utility of the present uses are demonstrated by the protection of spinal cord cells, tissues or organs from a spinal cord trauma by administering the above noted pharmaceutical composition at a time outside the therapeutic window of methylprednisone. A further example relates to the protection of brain cells, tissues, or organs from a stroke or brain trauma by administering the above noted pharmaceutical composition at a time outside the therapeutic window of recombinant tissue plasminogen activator.

The present invention further relates to a method for treating an injury to a mammalian cell, tissue or organ by administering to a mammal a pharmaceutical composition comprising an erythropoietin or a tissue protective cytokine wherein the pharmaceutical composition is administered at a time outside a therapeutic window recognized for currently approved therapeutics for the injury. Preferably, the pharmaceutical composition is administered prior to a therapeutic window recognized for a currently approved therapeutic for the injury, or after the therapeutic window recognized for the currently approved therapeutic for the injury. In particular the pharmaceutical composition may be administered to mammal at any at any two of the following three times: (1) prior to a therapeutic window recognized for a currently approved therapeutic for the trauma or injury, (2) within the therapeutic window recognized for a currently approved therapeutic for the trauma or injury, or (3) after the therapeutic window recognized for the currently approved therapeutic for the trauma or injury. Preferably, the pharmaceutical composition is administered a time after the therapeutic window recognized for currently approved therapeutics and at least one of the following times: (1) prior to a therapeutic window recognized for a currently approved therapeutic for the trauma or injury, or (2) within the therapeutic window recognized for a currently approved therapeutic for the trauma or injury. Additionally, the pharmaceutical composition may be administered at all three of the above noted times.

The present method may be practiced under the above noted conditions for the use proposed for the current invention as well.

### BRIEF DESCRIPTION OF THE FIGURES

FIG. 1 shows that asialo erythropoietin and carbamoylated erythropoietin administered as a single dose, have a prophylactic therapeutic effect when administered about 24 hours prior to the injury in a rat model of spinal cord injury.

FIG. 2 shows that erythropoietin, administered as a single dose, has a therapeutic effect when administered about 24 hours after the injury in a rat model of spinal cord injury.

FIG. 3 shows that carbamoylated erythropoietin, administered in a multiple dose regimen, has a therapeutic effect when administered up to at least about 72 hours following the injury in a rat model of spinal cord injury.

FIG. 4 shows that asialo erythropoietin, administered in a multiple dose regimen, has a therapeutic effect when administered up to about 24 hours following the injury in a rat model of spinal cord injury.

FIG. 5 shows that the prophylactic administration of a tissue protective cytokine, asialo erythropoietin, 1.5 or 4 hours prior to a middle cerebral artery occlusion in a rat model has a prophylactic therapeutic effect.

FIG. 6 shows that the administration of carbamoylated erythropoietin at 24 hours and 48 hours following middle cerebral artery occlusion has a therapeutic effect on rats paw placement using De Ryck's test.

FIG. 7 shows that the administration of carbamoylated erythropoietin at 24 hours and 48 hours following middle cerebral artery occlusion in rats reduces the number of foot faults in rats.

### DETAILED DESCRIPTION OF THE INVENTION

### I. Introduction

The methods of the invention provide for the local or systemic protection of cells, tissues and organs within a mammalian body or restoration or regeneration of dysfunction resulting from an injury through the administration of an erythropoietin or a tissue protective cytokine at a time outside of, i.e. before or after, the therapeutic window recognized for an approved therapeutic for that injury. As mentioned above, the ability of an erythropoietin or a tissue protective cytokine to provide these therapeutic effects outside of commonly recognized therapeutic windows offers the potential to prevent as well as treat a wide variety of conditions and diseases in individuals under conditions that previously would have been precluded because of the limited, currently approved therapeutic window. The invention is directed generally to the use of an erythropoietin or a tissue protective cytokine for the preparation of pharmaceutical compositions for the aforementioned purposes in which cellular function is maintained, promoted, enhanced, regenerated, or in any other way benefited by administration of the pharmaceutical composition outside of the therapeutic window of currently approved therapeutics for the injury to be treated. The invention is also directed to methods for maintaining, promoting, or regenerating cellular function by administering to a mammal an effective amount of an erythropoietin or a tissue protective cytokine as described herein.

The various methods of the invention utilize a pharmaceutical composition that at least includes erythropoietin or a tissue protective cytokine at an effective amount for the particular route of administration, time of administration, and duration of exposure to exert positive effects or benefits on responsive cells within a mammalian body. Where the target cell, tissues, or organs of the intended therapy require an erythropoietin or a tissue protective cytokine to cross an endothelial cell barrier, the pharmaceutical composition includes an erythropoietin or a tissue protective cytokine at a concentration which is capable, after crossing the endothelial cell barrier, of exerting its desirable effects upon the responsive cells. Molecules capable of interacting with the erythropoietin receptor and modulating the activity of the receptor, herein referred to generally as erythropoietin or erythropoietin receptor activity modulators, are useful in the context of the present invention. These molecules may be, for example, naturally-occurring, synthetic, or recombinant forms of erythropoietin molecules, as described below, or other molecules which may not necessarily resemble erythropoietin in any manner, except to modulate erythropoietin responsive cell activity, as described herein.

### II. Terminology

A. “Erythropoietin” is a glycoprotein hormone which in humans has a molecular weight of about 30-34 kDa. The mature protein has about 165 amino acids (SEQ. ID. NO. 5), and the oligosaccharide residues comprise about 40% of the weight of the molecule. Erythropoietin is defined by its erythropoietic effects: effects on the bone marrow—increased hematocrit (erythropoiesis), hyperactivation of platelets, procoagulant activity, and increased production of thrombocytes—or on the vasculature—vasoconstriction. See U.S. Pat. Nos. 4,703,008, 5,441,868, 5,547,933, 5,618,698, 5,621,080, 5,756,349, and 5,955,422. Recently, erythropoietin has also been attributed with tissue protective effects as previously disclosed in PCT Patent Application PCT/US00/10019, entitled Modulation of Excitable Tissue Function by Peripherally Administered Erythropoietin, which is incorporated herein by reference in its entirety. Erythropoietin can be obtained commercially, for example, under the trademarks of PROCRIT, available from Ortho Biotech Inc., Raritan, N.J., EPOGEN, available from Amgen, Inc., Thousand Oaks, Calif., and NEORECOROMON, erythropoietin beta, F. Hoffman-La Roche Ltd., Basel, Switzerland. Furthermore, a variety of host systems may be used for expression and production of recombinant erythropoietin, including, but not limited to, bacteria, yeast, insect, plant, and mammalian, including human, cell systems. For example, recombinant erythropoietin produced in bacteria, which do not glycosylate or sialate the product, can be used to produce non-glycosylated forms of erythropoietin. Alternatively, recombinant erythropoietin can be produced in other systems that do glycosylate, e.g., plants, and human cells.

Additionally, modified forms of erythropoietin are also useful in the methods of the present invention. Modified erythropoietines encompass chemical modifications and/or expression-system-mediated glycosylation modifications of naturally occurring, synthetic and recombinant forms of human and other mammalian erythropoietines. Various modified forms of erythropoietin have been described previously. Although these modified forms of erythropoietin were directed towards improving the erythropoietic activity of the molecule, such modification may also be suitable for purposes of the present method. These modified erythropoietines include, but are not limited to, enhanced forms of erythropoietin such as those with altered amino acids at the carboxy terminus described in U.S. Pat. No. 5,457,089 and in U.S. Pat. No. 4,835,260; erythropoietin isoforms with various numbers of sialic acid residues per molecule, such as described in U.S. Pat. No. 5,856,298; polypeptides described in U.S. Pat. No. 4,703,008; agonists described in U.S. Pat. No. 5,767,078; peptides which bind to the erythropoietin receptor as described in U.S. Pat. Nos. 5,773,569 and 5,830,851; and small-molecule mimetics as described in U.S. Pat. No. 5,835,382. Additionally, modified forms of erythropoietin having an in vivo half life greater than that of either naturally occurring or recombinant human erythropoietin are encompassed by the present uses and methods. In particular, erythropoietines having an extended half-life due to increased sialic acid residues as taught in U.S. Pat. No. 5,856,298, the addition of sugars as taught in EP0640619, the addition of polyethylene glycol (PEG) residues as taught in WO0102017, WO0032772, and U.S. Patent Application Serial No. 200301666566, the additions of proteins through fusion with erythropoietin as taught in U.S. Patent Application Serial Nos. 20040009902, 20030124115, and 20030113871 as well as U.S. Pat. No. 6,242,570, the modification of the naturally occurring glycosylation pattern of either recombinant or naturally occurring human erythropoietin as taught in PCT application number US/94/02957 and U.S. Patent Application Serial No. 20030077753, and/or mutations as taught in U.S. Patent Application Serial No. 20020081734 are included within the present invention. Modified erythropoietines include diglycosylated and pegylated erythropoietin conjugates taught in the following patent applications WO0102017, EP1064951, EP1345628, WO03029291, EP0640619, US2003077753, US20030120045 and U.S. Pat. Nos. 6,583,272 and 6,340,742. Examples of such long acting erythropoietines are ARANESP available from Amgen Inc., Thousand Oaks, Calif., USA, CERA available from F. Hoffmann-La Roche, Basel, Switzerland, and the diglycosylated and pegylated erythropoietines taught in WO03029291.

B. “Tissue Protective Cytokine” refers to cytokines that exhibit tissue protective effects and lack one or more of erythropoietin's effects on the bone marrow—increased hematocrit (erythropoiesis), hyperactivation of platelets, procoagulant activity, and increased production of thrombocytes—or on the vasculature—vasoconstriction (high blood pressure). The tissue protective cytokines may be chemically modified or recombinant forms of erythropoietin as previously disclosed in PCT/US03/20964 entitled Recombinant Tissue Protective Cytokines and Encoding Nucleic Acids Thereof for Protection, Restoration, and Enhancement of Responsive Cells, Tissues, and Organs; U.S. patent application Ser. No. 10/188,905, entitled Tissue Protective Cytokines for the Protection, Restoration, and Enhancement of Erythropoietin Responsive Cells, Tissues and Organs; and PCT Patent Application No. PCT/US01/49479 entitled Protection, Restoration, and Enhancement of Erythropoietin Cells, Tissues and Organs incorporated herein by reference. Preferably, the tissue protective cytokines are devoid of all erythropoietic activity.

The tissue protective cytokines may consist of chemically modified erythropoietines such as guanidation, amidination, carbamoylation (carbamoylation), triphenylation, acetylation, succinylation, nitration, or modification of argine, lysine, tyro sine, tryptophan, or cysteine residues or carboxyl groups, among other procedures such as limited proteolysis as disclosed in PCT/US01/49479. Additionally, tissue protective cytokines may consist of a recombinant erythropoietin as disclosed in PCT/US03/20964. The recombinant erythropoietin may consist of erythropoietin muteins. The disclosed mutations to erythropoietin may include substitutions, deletions, including internal deletions, additions, including additions yielding fusion proteins, or conservative substitutions of amino acid residues within and/or adjacent to the amino acid sequence, but that result in a “silent” change, and non-conservative amino acid changes and larger insertions and deletions. With both the chemically modified and recombinant erythropoietin, preferably the alterations occur within the erythropoietin amino acid sequence (SEQ ID NO:5) within four functional domains which affect receptor binding: VLQRY (SEQ ID NO:1) and/or TKVNFYAW (SEQ ID NO:2) and/or SGLRSLTTL (SEQ ID NO:3) and/or SNFLRG (SEQ ID NO:4). The skilled artisan can readily determine that a tissue protective cytokine useful for the purposes herein may have at least one of the aforementioned modifications, but may have more than one of the above modifications.

C. “Responsive cell” refers to a mammalian cell whose function or viability may be maintained, promoted, enhanced, regenerated, or in any other way benefited, by exposure to an erythropoietin. Non-limiting examples of such cells include neuronal, retinal, muscle, heart, lung, liver, kidney, small intestine, adrenal cortex, adrenal medulla, capillary endothelial, testes, ovary, pancreas, skin, bone and endometrial cells. In particular, responsive cells include, without limitation, neuronal cells; retinal cells: photoreceptor (rods and cones), ganglion, bipolar, horizontal, amacrine, and Müller cells; muscle cells; heart cells: myocardium, pace maker, sinoatrial node, sinus node, and junction tissue cells (atrioventricular node and bundle of His); lung cells; liver cells: hepatocytes, stellate, and Kupffer cells; kidney cells: mesangial, renal epithelial, and tubular interstitial cells; small intestine cells: goblet, intestinal gland (crypts) and enteral endocrine cells; adrenal cortex cells: glomerulosa, fasciculate, and reticularis cells; adrenal medulla cells: chromaffin cells; capillary cells: pericyte cells; testes cells: Leydig, Sertoli, and sperm cells and their precursors; ovary cells: Graffian follicle and primordial follicle cells; endometrial cells: endometrial stroma and endometrial cells; pancreas cell: islet of Langerhans, α-cells, β-cells, γ-cells, and δ-cells; skin cells; bone cells: osteoprogenitor, osteoclast and osteoblast cells; as well as the stem and endothelial cells present in the above listed organs. Moreover, such responsive cells and the benefits provided thereto by an erythropoietin or a tissue protective cytokine may be extended to provide protection or enhancement indirectly to other cells that are not directly responsive, or of tissues or organs which contain such non-responsive cells. These other cells, or tissues or organs which benefit indirectly from the enhancement of responsive cells present as part of the cells, tissue or organ as “associated” cells, tissues and organs. Thus, benefits of an erythropoietin or a tissue protective cytokine as described herein may be provided as a result of the presence of a small number or proportion of responsive cells in a tissue or organ, for example, excitable or neuronal tissue present in such tissue, or the Leydig cells of the testis, which makes testosterone. In one aspect, the responsive cell or its associated cells, tissues, or organs are not excitable cells, tissues, or organs, or do not predominantly comprise excitable cells or tissues.

D. “Injury” refers to the human diseases of the central nervous system or peripheral nervous system which have primarily neurological or psychiatric symptoms, ophthalmic diseases, cardiovascular diseases, cardiopulmonary diseases, respiratory diseases, kidney, urinary and reproductive diseases, bone diseases, skin diseases, gastrointestinal diseases and endocrine and metabolic abnormalities on which the erythropoietin and the tissue protective cytokines of the present invention have a therapeutic effect. In particular, such conditions and diseases include hypoxic conditions, which adversely affect excitable tissues, such as excitable tissues in the central nervous system tissue, peripheral nervous system tissue, or cardiac tissue or retinal tissue such as, for example, brain, heart, or retina/eye. Any condition which reduces the availability of oxygen to neuronal tissue, resulting in stress, damage, and finally, neuronal cell death, can be treated by the methods of the present invention. Generally referred to as hypoxia and/or ischemia, these conditions arise from or include, but are not limited to stroke, vascular occlusion, prenatal or postnatal oxygen deprivation, suffocation, choking, asthma, near drowning, carbon monoxide poisoning, smoke inhalation, trauma, including surgery and radiotherapy, asphyxia, epilepsy, hypoglycemia, chronic obstructive pulmonary disease, emphysema, adult respiratory distress syndrome, hypotensive shock, septic shock, anaphylactic shock, insulin shock, sickle cell crisis, cardiac arrest, dysrhythmia, nitrogen narcosis, and neurological deficits caused by heart-lung bypass procedures. Therefore, the method of the present invention may be used to treat or prevent damage to excitable tissue resulting from hypoxic conditions in a variety of conditions and circumstances. Non-limiting examples of such conditions and circumstances are provided in the table herein below.

| Cell, tissue | Dysfunction or | | |
|---|---|---|---|
| or organ | pathology | Condition or disease | Type |
| Heart | Ischemia | Coronary artery disease | Acute, chronic |
| | | | Stable, unstable |
| | | Myocardial infarction | |
| | | Angina | |
| | | Congenital heart disease | Valvular |
| | | | Cardiomyopathy |
| | | Prinzmetal angina | |
| | | Cardiac rupture | Aneurysmatic |
| | | | Septal perforation |
| | | Angiitis | |
| | Arrhythmia | Tachy-, bradyarrhythmia | Stable, unstable |
| | | Supraventricular, | Hypersensitive carotid sinus |
| | | ventricular | node |
| | | Conduction abnormalities | |
| | Congestive heart failure | Left, right, bi-ventricular | Cardiomyopathies, such as |
| | | | idiopathic familial, |
| | | | infective, metabolic, storage |
| | | | disease, deficiencies, |
| | | | connective tissue disorder, |
| | | | infiltration and granulomas, |
| | | | neurovascular |
| | | Myocarditis | Autoimmune, infective, |
| | | | idiopathic |
| | | Cor pulmonale | |
| | Blunt and penetrating | | |
| | trauma | | |
| | Toxins | Cocaine | |
| Vascular | Hypertension | Primary, secondary | |
| | Decompression | | |
| | sickness | | |
| | Fibromuscular | | |
| | hyperplasia | | |
| | Aneurysm | Dissecting, ruptured, | |
| | | enlarging | |
| Lungs | Obstructive | Asthma | |
| | | Chronic bronchitis, | |
| | | Emphysema and airway | |
| | | obstruction | |
| | | ARDS | |
| | Ischemic lung disease | Pulmonary embolism, | |
| | | Pulmonary thrombosis, | |
| | | Fat embolism | |
| | Environmental lung | | |
| | diseases | | |
| | Ischemic lung disease | Pulmonary embolism | |
| | | Pulmonary thrombosis | |
| | Interstitial lung disease | Idiopathic pulmonary | |
| | | fibrosis | |
| | Congenital | Cystic fibrosis | |
| | Cor pulmonale | | |
| | Trauma | | |
| | Pneumonia and | Infectious, parasitic, toxic, | |
| | pneumonitides | traumatic, burn, aspiration | |
| | Sarcoidosis | | |
| Pancreas | Endocrine | Diabetes mellitus, type I | Beta cell failure, |
| | | and II | dysfunction |
| | | | Diabetic neuropathy |
| | | Other endocrine cell | |
| | | failure of the pancreas | |
| | Exocrine | Exocrine pancreas failure | Pancreatitis |
| Bone | Osteopenia | Primary | Hypogonadism |
| | | secondary | immobilisation |
| | | | Postmenopausal |
| | | | Age-related |
| | | | Hyperparathyroidism |
| | | | Hyperthyroidism |
| | | | Calcium, magnesium, |
| | | | phosphorus and/or vitamin |
| | | | D deficiency |
| | Osteomyelitis | | |
| | Avascular necrosis | | |
| | Trauma | | |
| | Paget's disease | | |
| Skin | Alopecia | Areata | Primary |
| | | Totalis | Secondary |
| | | | Male pattern baldness |
| | Vitiligo | Localized | Primary |
| | | Generalized | Secondary |
| | Diabetic ulceration | | |
| | Peripheral vascular | | |
| | disease | | |
| | Burn injuries | | |
| Autoimmune | Lupus erythematodes, | | |
| disorders | Sjogren's syndrome, | | |
| | Rheumatoid arthritis, | | |
| | Glomerulonephritis, | | |
| | Angiitis | | |
| | Langerhan's | | |
| | histiocytosis | | |
| Eye | Optic neuritis | | |
| | Blunt and penetrating | | |
| | injuries, Infections, | | |
| | Sarcoid, Sickle C | | |
| | disease, Retinal | | |
| | detachment, Temporal | | |
| | arteritis | | |
| | Retinal ischemia, | | |
| | macular degeneration, | | |
| | retinal detachment, | | |
| | retinitis pigmentosa, | | |
| | arteriosclerotic | | |
| | retinopathy, | | |
| | hypertensive | | |
| | retinopathy, retinal | | |
| | artery blockage, retinal | | |
| | vein blockage, | | |
| | hypotension, and | | |
| | diabetic retinopathy. | | |
| Embryonic and | Asphyxia | | |
| fetal disorders | Ischemia | | |
| and pregnancy | Eclampsia | | |
| | Ischemic stroke | | |
| | hemorrhagic stroke | | |
| | brain trauma | | |
| | spinal cord trauma | | |
| | epilepsy | | |
| | convulsions | | |
| | chronic seizure disorder | | |
| CNS | Chronic fatigue | | |
| | syndrome, acute and | | |
| | chronic hypoosmolar | | |
| | and hyperosmolar | | |
| | syndromes, AIDS | | |
| | Dementia, | | |
| | Electrocution | | |
| | Asphyxia | | |
| | Multiple sclerosis | | |
| | Alzheimer's disease | | |
| | Parkinson's disease | | |
| | Cerebral palsy | | |
| | Age-related loss of | | |
| | cerebral function | | |
| | Memory loss | | |
| | ALS | | |
| | Seizure disorder | | |
| | Subacute sclerosing | | |
| | panencephalitis | | |
| | Encephalitis | Rabies, Herpes | |
| | Meningitis | | |
| | Subdural hematoma | | |
| | Nicotine addiction | | |
| | Drug abuse and | Cocaine, heroin, crack, | |
| | withdrawal | marijuana, LSD, PCP, | |
| | | poly-drug abuse, ecstasy, | |
| | | opioids, sedative | |
| | | hypnotics, amphetamines, | |
| | | caffeine, alcohol | |
| | Neuropsychiatric | Obsessive-compulsive | |
| | | disorders, mood disorders, | |
| | | anxiety disorders, | |
| | | depression, autism, | |
| | | attention deficit | |
| | | hyperactivity disorder, | |
| | | cognitive dysfunction. | |
| | Spinal stenosis, | | |
| | Transverse myelitis, | | |
| | Guillian Barre, Trauma, | | |
| | Nerve root | | |
| | compression, Tumoral | | |
| | compression, Heat | | |
| | stroke | | |
| | tuberous sclerosis, | | |
| | Wilson's Disease, | | |
| | cerebral and | | |
| | progressive | | |
| | supranuclear palsy, | | |
| | Guam disease, Lewy | | |
| | body dementia, | | |
| | Huntington's disease, | | |
| | myotonic dystrophy, | | |
| | Freidrich's ataxia and | | |
| | other ataxias, Gilles de | | |
| | la Tourette's syndrome | | |
| | prion diseases | spongiform | |
| | | encephalopathies, | |
| | | Creutzfeldt-Jakob disease | |
| | Cardiopulmonary | | |
| | bypass | | |
| ENT | Tinnitus | | |
| | Meuniere's syndrome | | |
| | Hearing loss | | |
| | Traumatic injury, | | |
| | barotraumas | | |
| Kidney | Renal failure | Acute, chronic | Vascular/ischemic, |
| | | | interstitial disease, diabetic |
| | | | kidney disease, nephrotic |
| | | | syndromes, infections |
| | Henoch-Schonlein | | |
| | Purpura | | |
| | transplant | | |
| Striated muscle | Autoimmune disorders | Myasthenia gravis | |
| | | Dermatomyositis | |
| | | Polymyositis | |
| | Myopathies | Inherited metabolic, | |
| | | endocrine and toxic | |
| | Heat stroke | | |
| | Crush injury | | |
| | Rhabdomylosis | | |
| | Mitochondrial disease | | |
| | Infection | Necrotizing fasciitis | |
| Sexual | Central and peripheral | Impotence secondary to | |
| dysfunction | | medication | |
| Liver | Hepatitis | Viral, bacterial, parasitic | |
| | Ischemic disease | | |
| | Cirrhosis, fatty liver | | |
| | Infiltrative/metabolic | | |
| | diseases | | |
| Gastrointestinal | Ischemic bowel disease | | |
| | Inflammatory bowel | | |
| | disease | | |
| | Necrotizing | | |
| | enterocolitis | | |
| Organ | Treatment of donor and | | |
| transplantation | recipient | | |
| Reproductive | Infertility | Vascular | |
| tract | | Autoimmune | |
| | | Uterine abnormalities | |
| | | Implantation disorders | |
| Endocrine | Glandular hyper- and | | |
| | hypofunction | | |

E. “Therapeutic window” is the time period relative to the initial injury during which administration of a therapeutic produces a demonstrable clinical effect. For example, recombinant tissue-type plasminogen activator (rt-PA) is an approved therapeutic for the treatment of ischemic stroke. It has demonstrated clinical effect in patients up to three (3) hours following their ischemic stroke. Therefore, rt-PA has a therapeutic window of three (3) hours in stroke. In addition to the particular injury, the therapeutic window is based upon the severity of the injury—the therapeutic window may differ between a slight injury and a severe injury. To accurately compare the therapeutic windows of therapeutics, the injury and severity of the injury must be taken into account. The therapeutic window of various therapeutic compounds may be determined by referencing the manufacturers labeling as well as the Physicians Desk Reference published by Medical Economics Company, Inc. or other generally recognized equivalents.

The therapeutic window may be further subdivided into a “Prophylactic Therapeutic Window” that refers to the time period prior to an injury during which the administration of a therapeutic will provide protection against or ameliorate damage resulting from the injury. When an individual suffers an acute injury, including, but not limited to, surgery, trauma (blunt, etc.), stroke, poisoning, it may be easier to determine the therapeutic window than in a neurodegenerative condition or progressive disease. However, in the case of the a neurodegenerative condition or progressive disease, the therapeutic window may be the period from the initiation of the next phase or stage of the neurodegenerative condition or progressive disease. Furthermore, administration is considered outside of a therapeutic window if it occurs either prior to or after that therapeutic window.

F. “Approved Therapeutic” refers to a therapeutic approved for treatment of a particular injury by a regulatory agency of the Federal or a state government or listed in the U.S. Pharmacopeia or other generally recognized foreign pharmacopeia for use in animals and more particularly in humans. As examples, the following therapeutics would be considered approved therapeutics within their particular indications: rt-PA (sold under the name ACTIVASE, by Genentech Inc., South San Francisco, Calif.) for use in the treatment of ischemic strokes, corticosteroids such as methylprednisone for use in the treatment of spinal cord injury, and thrombolytics such as rt-PA (TNKase, Genentech, South San Francisco, Calif. and RETAVASE, Centocor, Inc., Malvern, Pa.) and streptokinase (STREPASE, AstraZeneca LP, Wilmington, Del.).

### II. Method of Treatment

The present invention provides for the use of erythropoietin and/or the above disclosed tissue protective cytokines for the preparation of a pharmaceutical composition that may be administered outside the therapeutic window of previously approved therapeutics for a particular injury to responsive cells, tissues, or organs. The methods of the present invention provide that the pharmaceutical composition may be administered prior to the injury (prophylactic use), or after the therapeutic window for a previously approved therapeutic has lapsed (extended use). Accordingly, this invention generally provides therapeutic or prophylactic treatment of the consequences of mechanical trauma or of human diseases. Therapeutic or prophylactic treatment for diseases, disorders or conditions of the CNS and/or peripheral nervous system are preferred. Therapeutic or prophylactic treatment for diseases, disorders or conditions including but not limited to those having an ophthalmic, cardiovascular, cardiopulmonary, respiratory, kidney, urinary, reproductive, gastrointestinal, endocrine, or metabolic component is provided.

A. Prophylactic Use.

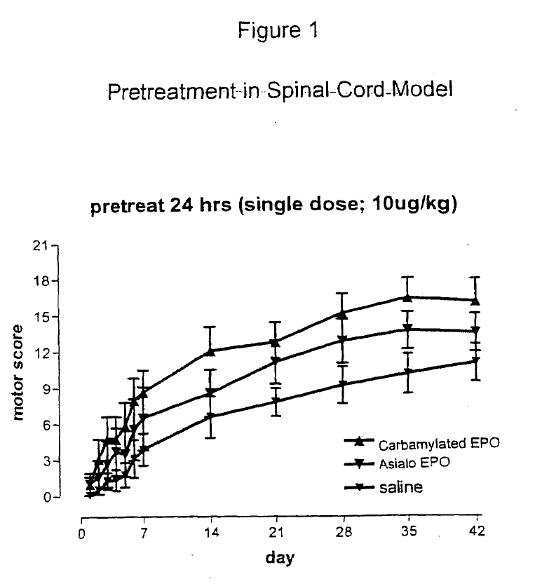

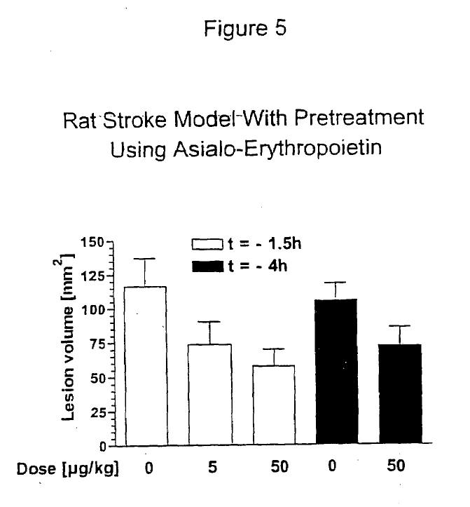

As noted above, the pharmaceutical compositions including erythropoietin and/or tissue protective cytokines of the present invention, permit them to be used prophylactically by those engaged in activities that may result in injuries to responsive tissues, i.e. head, spinal cord, eye, heart, and kidney injuries. This prophylactic use is demonstrated in FIG. 1, where pretreatment with a single dose of a tissue protective cytokine such as asialo erythropoietin or carbamoylated erythropoietin resulted in enhanced recovery in a rat model of spinal cord injury. Additionally, FIG. 5 further demonstrates a similar effect in a middle cerebral artery occlusion model in rats. A tissue protective cytokine, asialo erythropoietin, administered at 1.5 or 4 hours following occlusion of the middle cerebral artery demonstrated a therapeutic effect by reducing the infarct volume resulting from the occlusion.

Thus, prior to engaging in such hazardous activities, one could take a dose of a pharmaceutical composition containing erythropoietin and/or a tissue protective cytokine sufficient to either prevent (i.e. delaying the onset of, inhibiting, or stopping), protect against, or mitigate the damage resulting from an injury to a responsive cell, tissue, or organ. In particular, this method of treatment may have application in various professions susceptible to injury such as, but not limited to, professional athletes (divers, race car drivers, football players, etc.), military personnel (soldiers, paratroopers), emergency personnel (police, fire, EMS, and disaster relief personnel), stuntmen, and construction workers. Additionally, the prophylactic use of tissue protective cytokines is contemplated in such recreational endeavors including, but not limited to, rock climbing, rappelling, sky diving, racing, bicycling, football, rugby, baseball, and diving that pose a risk of injury.

Additionally, the pharmaceutical compositions of the present invention may be used prophylactically to prepare an individual for surgery in an effort to limit the trauma associated with the surgical procedure or aide in the recovery of the individual from the surgical procedure. Although the present method of treatment using pharmaceutical compositions containing erythropoietin and/or tissue protective cytokines provides a prophylactic use for surgical procedures, it may be particularly useful in procedures that induce temporary ischemic events including, but not limited to, bypass procedures (coronary bypass), angioplasty procedures, amputations, and transplantations, as well as, those performed directly upon responsive cells, tissues, or organs such as brain and spinal cord surgery, and open heart procedures. Such procedures may involve the use of cardiopulmonary (heart lung) bypass.

Preferably, a tissue protective cytokine, more preferably a tissue protective cytokine completely devoid of erythropoietic effects, would be administered for this prophylactic administration to avoid complications due to the erythropoietic effects of erythropoietin and, within the realm of professional sports, the perception of enhanced performance due to the administration of the compound. However, use of erythropoietin is contemplated as well provided that the use is monitored so as to avoid a harmful increase in red blood cells or its illicit use. Additionally, a single dose prophylactic administration is preferred although different dosing regimens are contemplated by the present invention and may be dictated by the particular endeavor.

The pharmaceutical composition of the present invention may be administered at about 1 to 24 hours prior to the injury, more preferably at about 6 to 18 hours prior to the injury or trauma, and most preferably at least about 24 hours prior to the injury. Skilled practitioners will recognize that modified forms of erythropoietin or tissue protective cytokines that exhibit extended half-lives, such as those disclosed within PCT Application No. PCT/US0328073 entitled Long Acting Erythropoietines that Maintain Tissue Protective Activity of Endogenous Erythropoietin, incorporated herein by reference, may extend the prophylactic therapeutic window for the pharmaceutical composition even further.

B. Extended Use.

Furthermore, the tissue protective cytokines of the present invention also have a large therapeutic window after the injury occurs. Often an injured individual may not receive treatment shortly after (0-3 hrs) the injury due to a failure to recognize that the injury has occurred, inability to reach medical treatment, or need to address other injuries prior to addressing the injury to the responsive cells, tissues or organs. Thus, certain therapeutics are effectively removed from the array of treatments available to patients because the patients rarely receive treatment within the therapeutic window for these therapeutics. For example, tissue-type plasminogen activator (rt-PA) used in ischemic stroke patients has a therapeutic window of only 3 hours. Other approved therapeutics suffer from the same limitations including but not limited to methylprednisone, a corticosteroid used in the treatment of spinal cord injuries, must be administered within eight (8) hours from the initial injury; and thrombolytics such as rt-PA or streptokinase must be administered within three (3) to six (6) hours of myocardial infarction. Thus, it is contemplated that a pharmaceutical composition comprised of erythropoietin and/or a tissue protective cytokine may be used to provide therapeutic effects despite extended delays in initiating treatment.

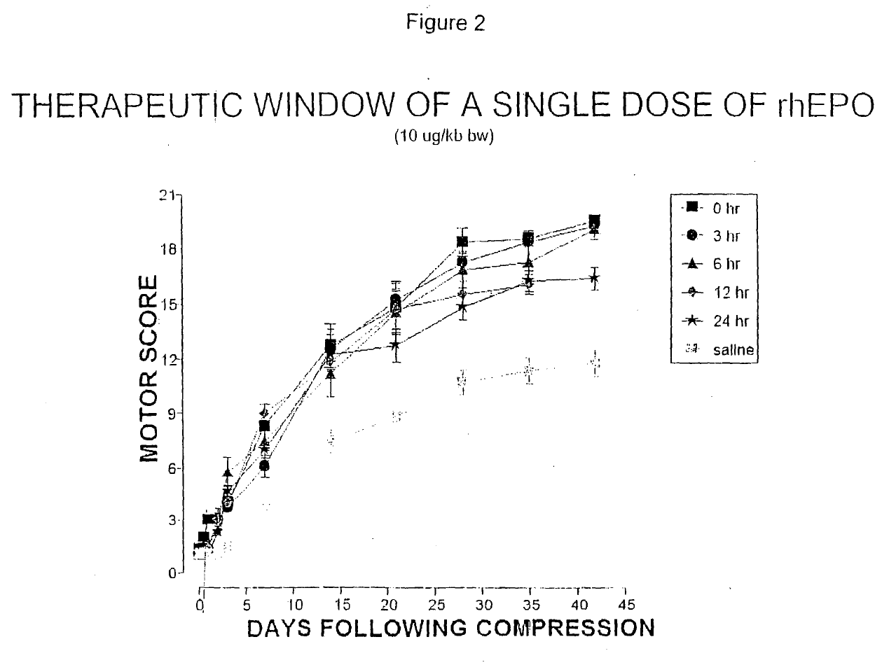

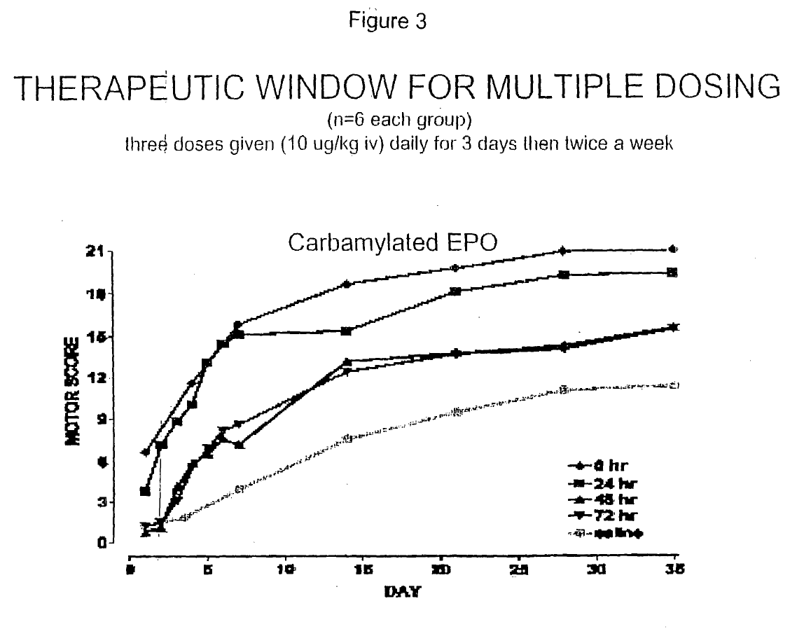

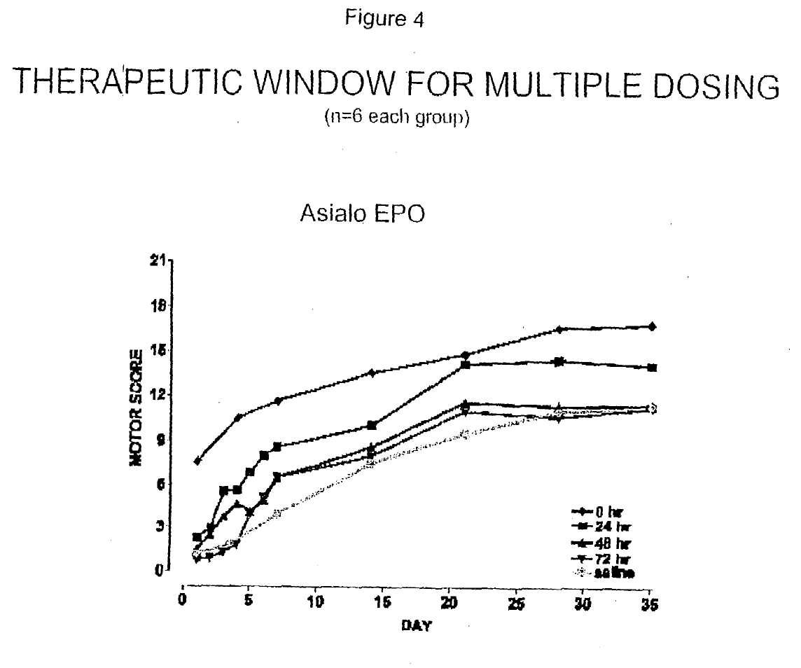

As noted within FIGS. 2-4, the pharmaceutical compositions of the present invention may extend the therapeutic window substantially beyond the occurrence of the initial injury or trauma. FIG. 2 demonstrates that a single dose of recombinant erythropoietin administered twenty-four (24) hours after the injury in the rat model of spinal cord injury model is still effective. FIGS. 3-4 also show that a multiple dosing regimen of tissue protective cytokines, asialo erythropoietin and carbamoylated erythropoietin, initiated 48 hours and 72 hours, respectively, following injury in the rat spinal cord injury model still remained effective.

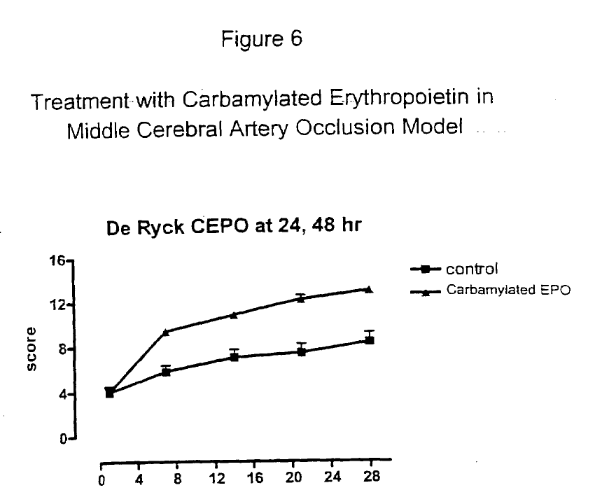

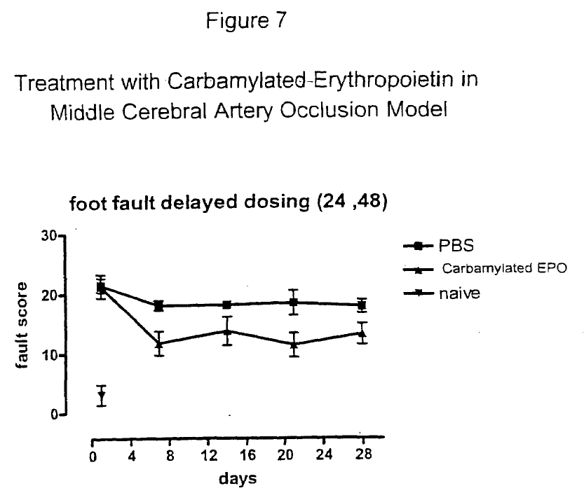

Additionally, FIGS. 6-7, demonstrate that administration of a tissue protective cytokine, carbamoylated erythropoietin, at 24 hours following a middle cerebral artery occlusion still has a positive effect on the behavior of rats. This result is particularly surprising given that 24 hours after a stroke it is established in the literature that most of the damage resulting from the stroke is irreversible.

In particular, the present method of treatment would be helpful in combat or disaster situations where those injured may not be able to receive treatment within a timely fashion after being injured.

It is contemplated that depending upon the amount of time that has lapsed since the injury occurred, one of ordinary skill in the art may need to adjust the dosing regimen (single dose or multiple dose), amount of dose (higher doses may be required as time passes), or route of administration (more direct routes may be warranted—intravenous administration as opposed subcutaneous) to achieve the desired therapeutic effect. According to the methods of the present invention the pharmaceutical compositions may be administered 8 to 168 hours following the injury, preferably about 12 to 72 hours following the injury. Although this example relates to stroke, it is exemplary of the pharmaceutical compositions therapeutic window with regard to additional indications such as spinal cord injury. Thus, the use of erythropoietin and/or tissue protective cytokines within a pharmaceutical composition permits these compositions to better suited address the realities of preventing and treating injuries to responsive cells, tissues, and organs.

C. Treatment Regime.

Skilled practitioners recognize that the methods and uses of the present invention may be combined for enhanced effect.

It is contemplated that the pharmaceutical compositions of the present invention may be administered at multiple time periods with respect to the therapeutic window of previously approved therapeutics to optimize its therapeutic effect. Thus, the pharmaceutical composition comprised of erythropoietin and/or tissue protective cytokines may be administered at least any two of the following times: prior to the injury, at about the time of the injury or within the therapeutic window of previously approved therapeutics, or beyond the therapeutic window of previously approved therapeutics. The pharmaceutical composition may also be administered within all three time periods. Such a treatment regime may be preferential in situations such as combat wherein the pharmaceutical composition can be administered to a soldier prior to combat and then administered again on the battlefield if the soldier is wounded or at a later period if the circumstances do not permit the timely treatment of the wounded soldier.

Additionally, one of ordinary skill in the art would recognize that some versions of erythropoietin or tissue protective cytokines may be better suited to either prophylactic or extended uses. Thus, one would recognize the benefit of using one erythropoietin or tissue protective cytokine for a prophylactic administration and another erythropoietin or tissue protective cytokine for extended administration. For example, prior to a surgery an asialo erythropoietin may be administered prophylactically and subsequent to the surgery another erythropoietin or tissue protective cytokine may be administered to assist in the patients recovery.

Additionally, skilled practitioners will recognize the benefit in administering other therapeutics simultaneously with the present pharmaceutical compositions for the purposes of treating other aspects of the individuals injury or achieving synergistic effects—enhancing the efficacy of the other therapeutics, enhancing the efficacy of the erythropoietin or tissue protective cytokine.

In another aspect of the present invention, a pharmaceutical composition according to the present invention may include an erythropoietin and/or tissue protective cytokine in a formulation with at least one small molecule that exhibits tissue protective functionality. Suitable small molecules include, but are not limited to, steroids (e.g., lazaroids and glucocorticoids), antioxidants (e.g., coenzyme Q₁₀, alpha lipoic acid, and NADH), anticatabolic enzymes (e.g., glutathione peroxidase, superoxide dimutase, catalase, synthetic catalytic scavengers, as well as mimetics), indole derivatives (e.g., indolamines, carbazoles, and carbolines), nitric acid neutralizing agents, adenosine/adenosine agonists, phytochemicals (flavonoids), herbal extracts (ginkgo biloba and turmeric), vitamins (vitamins A, E, and C), oxidase electron acceptor inhibitors (e.g., xanthine oxidase electron inhibitors), minerals (e.g., copper, zinc, and magnesium), non-steroidal anti-inflammatory drugs (e.g., aspirin, naproxen, and ibuprofen), and combinations thereof. Additionally agents including, but not limited to, anti-inflammatory agents (e.g., corticosteroids, prednisone and hydrocortisone), glucocorticoids, steroids, non-steroidal anti-inflammatory drugs (e.g., aspirin, ibuprofen, diclofenac, and COX-2 inhibitors), beta-agonists, anticholinergic agents and methyl xanthines), immunomodulatory agents (e.g., small organic molecules, a T cell receptor modulators, cytokine receptor modulators, T-cell depleting agents, cytokine antagonists, monokine antagonists, lymphocyte inhibitors, or anti-cancer agents), gold injections, sulphasalazine, penicillamine, anti-angiogenic agents (e.g., angiostatin, TNF-α antagonists (e.g., anti-TNFα antibodies), and endostatin), dapsone, psoralens (e.g., methoxalen and trioxsalen), anti-malarial agents (e.g., hydroxychloroquine), anti-viral agents, and antibiotics (e.g., erythromycin and penicillin) may be used in conjunction with the current pharmaceutical compositions.

Furthermore, the current pharmaceutical compositions may be used in combination with previously approved therapeutics including but not limited to rt-PA, methylprednisone, and thrombolytics in a synergistic manner to extend the therapeutic window of those compounds.

### IV. Pharmaceutical Compositions

In one embodiment, such a pharmaceutical composition of erythropoietin or tissue protective cytokine may be administered systemically to protect or enhance the target cells, tissue or organ. Such administration may be parenterally, via inhalation, transdermally, or transmucosally, e.g., orally, nasally, rectally, intravaginally, sublingually, or submucosally. Preferably, administration is parenteral, e.g., via intravenous or intraperitoneal injection, and also including, but not limited to, intra-arterial, intramuscular, intradermal and subcutaneous administration.

For other routes of administration, such as by use of a perfusate, injection into an organ, or other local administration, a pharmaceutical composition will be provided which results in similar levels of a tissue protective cytokine as described above. A level of about 15 pM-30 nM in the tissues is preferred.

The pharmaceutical compositions of the invention may comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal and pulmonary administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. Alternatively, inhalation of compounds directly into the lungs may be accomplished by inhalation deeply or installation through a mouthpiece into the oropharynx. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered into the nasal cavity directly or into the lungs via the nasal cavity or oropharynx.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets. In one embodiment, an autoinjector comprising an injectable solution of an erythropoietin may be provided for emergency use by ambulances, emergency rooms, and battlefield situations, and even for self-administration in a domestic setting, particularly where the possibility of traumatic amputation may occur, such as by imprudent use of a lawn mower.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

Suppositories generally contain active ingredient in the range of 0.5% to 10% by weight; oral formulations preferably contain 10% to 95% active ingredient.

A perfusate composition may be provided for use in transplanted organ baths, for in situ perfusion, or for administration to the vasculature of an organ donor prior to organ harvesting. Such pharmaceutical compositions may comprise levels of erythropoietin, tissue protective cytokines, or a form of either erythropoietin or tissue protective cytokines not suitable for acute or chronic, local or systemic administration to an individual, but will serve the functions intended herein in a cadaver, organ bath, organ perfusate, or in situ perfusate prior to removing or reducing the levels of the erythropoietin contained therein before exposing or returning the treated organ or tissue to regular circulation. The erythropoietin for this aspect of the invention may be any erythropoietin, such as naturally-occurring forms such as human erythropoietin, or any of tissue protective cytokines herein above described, such as asialo erythropoietin and phenylglyoxal-erythropoietines, as non-limiting examples.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, for example, the pharmaceutical composition can be delivered in a controlled-release system. For example, it may be administered using intravenous infusion, an implantable osmotic pump, a transdermal patch, liposomes, or other modes of administration. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); WO 91/04014; U.S. Pat. No. 4,704,355; Lopez-Berestein, ibid., pp. 317-327; see generally ibid.). In another embodiment, polymeric materials can be used (see Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, J. Macromol. Sci. Rev. Macromol. Chem. 23:61, 1953; see also Levy et al., 1985, Science 228:190; During et al., 1989, Ann. Neurol. 25:351; Howard et al., 1989, J. Neurosurg. 71:105).

In yet another embodiment, a controlled release system can be placed in proximity of the therapeutic target, i.e., the target cells, tissue or organ, thus requiring only a fraction of the systemic dose (see, e.g., Goodson, pp. 115-138 in Medical Applications of Controlled Release, vol. 2, supra, 1984). Other controlled release systems are discussed in the review by Langer (1990, Science 249:1527-1533).

In another embodiment, the pharmaceutical composition, as properly formulated, can be administered by nasal, oral, rectal, vaginal, or sublingual administration.

In a specific embodiment, it may be desirable to administer erythropoietin and/or the tissue protective cytokines of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers.

### V. Dosing

The activity (in units) of erythropoietin and erythropoietin-like molecules is traditionally defined based on its effectiveness in stimulating red cell production in rodent models (and as derived by international standards of erythropoietin). One unit (U) of regular erythropoietin (MW of ˜34,000) is about 8 ng of protein (1 mg protein is approximately 125,000 U). However, as the effect on erythropoiesis is incidental to the desired activities herein and may not necessarily be a detectable property of certain of the tissue protective cytokines of the invention, a definition of activity of certain tissue protective cytokines of the invention based on erythropoietic activity is inappropriate. Thus, as used herein, the activity unit of erythropoietin or the tissue protective cytokines is defined as the amount of protein required to elicit the same activity in neural or other responsive cellular systems as is elicited by WHO international standard erythropoietin in the same system. The skilled artisan will readily determine the units of a non-erythropoietic erythropoietin or related tissue protective cytokine molecule following the guidance herein.

Selection of the preferred effective dose will be readily determinable by a skilled artisan based upon considering several factors, which will be known to one of ordinary skill in the art. Such factors include the particular form of erythropoietin or the tissue protective cytokine, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether administration is acute or chronic, concomitant medications, and other factors well known to affect the efficacy of administered pharmaceutical agents. Thus the precise dosage should be decided according to the judgment of the practitioner and each patient's circumstances, e.g., depending upon the condition and the immune status of the individual patient, and according to standard clinical techniques.

While the preferred recipient of a tissue protective cytokine for the purposes herein throughout is a human, the methods herein apply equally to other mammals, particularly domesticated animals, livestock, companion, and zoo animals. However, the invention is not so limiting and the benefits can be applied to any mammal.

In another aspect of the invention, methods and compositions for enhancing the viability of cells, tissues or organs which are not isolated from the vasculature by an endothelial cell barrier are provided by exposing the cells, tissue or organs directly to a pharmaceutical composition comprising erythropoietin or a tissue protective cytokine, or administering or contacting a pharmaceutical composition containing erythropoietin or a tissue protective cytokine to the vasculature of the tissue or organ. Enhanced activity of responsive cells in the treated tissue or organ is responsible for the positive effects exerted.

As described above, the invention is based, in part, on the discovery that erythropoietin molecules can be transported from the luminal surface to the basement membrane surface of endothelial cells of the capillaries of organs with endothelial cell tight junctions, including, for example, the brain, retina, and testis. Thus, responsive cells across the barrier are susceptible targets for the beneficial effects of erythropoietin or tissue protective cytokines, and others cell types or tissues or organs that contain and depend in whole or in part on responsive cells therein are targets for the methods of the invention. While not wishing to be bound by any particular theory, after transcytosis of erythropoietin or the tissue protective cytokine, erythropoietin or the tissue protective cytokine can interact with an erythropoietin receptor on a responsive cell, for example, neuronal, retinal, muscle, heart, lung, liver, kidney, small intestine, adrenal cortex, adrenal medulla, capillary endothelial, testes, ovary, or endometrial cell, and receptor binding can initiate a signal transduction cascade resulting in the activation of a gene expression program within the responsive cell or tissue, resulting in the protection of the cell or tissue, or organ, from damage, such as by toxins, chemotherapeutic agents, radiation therapy, hypoxia, etc. Thus, methods for protecting responsive cell-containing tissue from injury or hypoxic stress, and enhancing the function of such tissue are described in detail herein below.

In the practice of one embodiment of the invention, a mammalian patient is undergoing systemic chemotherapy for cancer treatment, including radiation therapy, which commonly has adverse effects such as nerve, lung, heart, ovarian or testicular damage. Administration of a pharmaceutical composition comprising erythropoietin and/or a tissue protective cytokine as described above is performed prior to and during chemotherapy and/or radiation therapy, to protect various tissues and organs from damage by the chemotherapeutic agent, such as to protect the testes. Treatment may be continued until circulating levels of the chemotherapeutic agent have fallen below a level of potential danger to the mammalian body.

In another embodiment of the invention, in any surgical procedure, such as in cardiopulmonary bypass surgery, a naturally-occurring erythropoietin or a tissue protective cytokine of the invention can be used. In one embodiment, administration of a pharmaceutical composition comprising erythropoietin and/or tissue protective cytokines as described above is performed prior to, during, and/or following the bypass procedure, to protect the function of brain, heart, and other organs.

In another embodiment of the invention, a surgical procedure to repair a heart valve required temporary cardioplegia and arterial occlusion. Prior to surgery, the patient was infused with a tissue protective cytokine, 4 μg of carbamoylated asialo erythropoietin per kg body weight. Such treatment prevented hypoxic ischemic cellular damage, particularly after reperfusion.

In the foregoing examples in which an erythropoietin and/or a tissue protective cytokine of the invention is used for ex-vivo applications, or to treat responsive cells such as neuronal tissue, retinal tissue, heart, lung, liver, kidney, small intestine, adrenal cortex, adrenal medulla, capillary endothelial, testes, ovary, or endometrial cells or tissue, the invention provides a pharmaceutical composition in dosage unit form adapted for protection or enhancement of responsive cells, tissues or organs distal to the vasculature which comprises an amount within the range from about 1 pg to 5 mg, 500 pg to 5 mg, 1 ng to 5 mg, 500 ng to 5 mg, 1 μg to 5 mg, 500 μg to 5 mg, or 1 mg to 5 mg of a tissue protective cytokine, and a pharmaceutically acceptable carrier. In a preferred embodiment, the amount of an erythropoietin or a tissue protective cytokine is within the range from about 1 pg to 1 mg. In a preferred embodiment, the formulation contains tissue protective cytokines that are non-erythropoietic.

Furthermore, this restorative aspect of the invention is directed to the use of any erythropoietines and/or tissue protective cytokines herein for the preparation of a pharmaceutical composition for the restoration of cellular, tissue or organ dysfunction, wherein treatment is initiated after, and well after, the initial insult responsible for the dysfunction. Moreover, treatment using erythropoietin and/or tissue protective cytokines of the invention can span the course of the disease or condition during the acute phase as well as a chronic phase.

In the instance wherein the pharmaceutical composition contains an erythropoietin, in a preferred embodiment, it may be administered systemically at a dosage between about 1 μg and about 100 μg/kg body weight, preferably about 5-50 μg/kg-body weight, most preferably about 10-30 μg/kg-body weight, per administration. This effective dose should be sufficient to achieve serum levels of erythropoietin greater than about 10,000, 15,000, or 20,000 mU/ml (80, 120, or 160 ng/ml) of serum after erythropoietin administration. Such serum levels may be achieved at about 1, 2, 3, 4, 5, 6, 7, 8, 9, or 10 hours post-administration. Such dosages may be repeated as necessary. For example, administration may be repeated daily, as long as clinically necessary, or after an appropriate interval, e.g., every 1 to 12 weeks, preferably, every 1 to 3 weeks. In one embodiment, the effective amount of erythropoietin and a pharmaceutically acceptable carrier may be packaged in a single dose vial or other container. In another embodiment, the tissue protective cytokines, which are capable of exerting the activities described herein but not causing an increase in hemoglobin concentration or hematocrit, are used. Such tissue protective cytokines are preferred in instances wherein the methods of the present invention are intended to be provided chronically. In another embodiment, an erythropoietin is given at a dose greater than that necessary to maximally stimulate erythropoiesis. As noted above, a tissue protective cytokine of the invention does not necessarily have erythropoietic activity, and therefore the above dosages expressed in hematopoietic units are merely exemplary for erythropoietines; herein above weight equivalents for dosages are provided which are applicable to tissue protective cytokines.

In the following examples, various animal models and in-vitro tests of the present methods of treatment are provided to demonstrate the effectiveness of the administration of pharmaceutical compositions comprising either erythropoietin or tissue protective cytokines outside of the therapeutic window of previously approved therapeutics. The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### Example 1

### Spinal Cord Injury Model

Rat Spinal Cord Compression Testing of Erythropoietin and Tissue Protective Cytokines

Female Wistar rats weighing 180-300 g were used in this study. The animals were fasted for 12 h before surgery, and were humanely restrained and anesthetized with an intraperitoneal injection of thiopental sodium (40 mg/kg-bw). After infiltration of the skin (bupivacaine 0.25%), a complete single level (T-3) laminectomy was performed through a 2 cm incision with the aid of a dissecting microscope. Traumatic spinal cord injury was induced by the extradural application of a temporary aneurysm clip exerting a 0.6 newton (65 grams) closing force on the spinal cord for 1 minute. After removal of the clip, the skin incision was closed and the animals allowed to recover fully from anesthesia and returned to their cages. The rats were monitored continuously and bladder palpation provided at least twice daily until spontaneous voiding resumed.

Motor neurological function of the rats was evaluated by use of the locomotor rating scale of Basso et al. 1995 J Neurotrauma 12:1-21. In this scale, animals are assigned a score ranging from 0 (no observable hindlimb movements) to 21 (normal gait). The rats were tested for functional deficits at 1, 12, 24, 48, 72 hours and then at 1 week after injury by the same examiner who is blind to the treatment each animal receives.

A. Prophylactic Therapeutic Window of Tissue Protective Cytokines.

18 animals were randomly divided into three groups. Animals in the control Group (I) (n=6) received normal saline (via intravenous injection) 24 hours prior to surgery. Group (II) (n=6) received asialo erythropoietin (10 micrograms/kg-bw iv), made in accordance with the protocols set forth in PCT/US01/49479, 24 hours prior to surgery; and group (III) (n=6) received a carbamoylated erythropoietin (10 micrograms/kg-bw iv, group), made in accordance with the protocols set forth in PCT/US01/49479, 24 hours prior to surgery.

FIG. 1 is a graph demonstrating the locomotor ratings of the rats recovering from the spinal cord trauma over a period of forty-two days. As can be seen from the graph, the rats that were given asialo erythropoietin (group II) or carbamoylated erythropoietin (group III) 24 hours prior to the surgery recovered from the injury more readily and demonstrated better overall recovery from the injury than the control rats. Similar results are expected from the therapeutic treatment with additional tissue protective cytokines referred to in the present disclosure.

B. Therapeutic Window of Tissue Protective Cytokines

### 1. Therapeutic Window of Single Dose

A spinal cord injury protocol as outlined above was used for the present example. While regulating the body temperature of the rat, a laminectomy was performed. An aneurysm clip was applied to the T3 vertebrae of the rat at 58 grams of force for 60 seconds. This was done in such a manner as to avoid affecting the paraspinal arteries. The rats were divided into groups and each of these groups was given the single dose of rhEPO (10 μg/kg bw) intravenously at different points in time after the injury, 0-24 hrs. Subsequently, the motor scores of the rats were monitored for 45 days in accordance with the protocol set forth above.

FIG. 2 shows that the rhEPO had a tissue protective effect even when the single dose of rhEPO was not administered until 24 hrs after the initial injury.

### 2. Therapeutic Window of Multiple Doses

A spinal cord injury protocol as outlined above was used for the present example. While regulating the body temperature of the rat, a laminectomy was performed. An aneurysm clip was applied to the T3 vertebrae of the rat at 58 grams of force for 60 seconds. This was done in such a manner as to avoid affecting the paraspinal arteries. After the trauma was applied, the rats were treated using tissue protective cytokines, carbamoylated and asialo erythropoietin, on a multiple dose regime (three doses (10 μg/kg bw) were given intravenously the first three days, and then twice a week following). The rats were divided into groups (N=6) and each of these groups started the multiple dose regime of tissue protective cytokines at different points in time after the injury, 0-72 hrs. Subsequently, the motor scores of the rats were monitored for 45 days in accordance with the protocol set forth above.

FIGS. 3 and 4 show that these tissue protective cytokines exhibit a tissue protective effect even when the compound was not administered shortly (0-6 hrs) after the initial injury. Specifically, FIG. 3 shows that the carbamoylated erythropoietin had a therapeutic effect even when it was not administered until 72 hrs after the injury. Similarly, FIG. 4 demonstrates that asialo erythropoietin has a therapeutic effect when administered 24 hrs after the injury.

### Example 2

### Middle Cerebral Artery Occlusion (MCAO) Studies

Male Crl:CD(SD)BR rats weighing 250-280 g were obtained from Charles River, Calco, Italy. Surgery was performed on these rats in accordance with the teachings of Brines, M. L., Ghezzi, P., Keenan, S., Agnello, D., de Lanerolle, N. C., Cerami, C., Itri, L. M., and Cerami, A. 2000 Erythropoietin crosses the blood-brain barrier to protect against experimental brain injury Proc Natl Acad Sci USA 97:10526-10531. Briefly, the rats were anesthetized with chloral hydrate (400 mg/kg-bw, i.p.), the carotid arteries were visualized, and the right carotid was occluded by two sutures and cut. A burr hole adjacent and rostral to the right orbit allowed visualization of the MCA, which was cauterized distal to the rhinal artery. To produce a penumbra (border zone) surrounding this fixed MCA lesion, the contralateral carotid artery was occluded for 1 hour by using traction provided by a fine forceps and then re-opened.

A. Prophylactic Therapeutic Window of Tissue Protective Cytokines.

Sprague Dawley rats were subjected to the above noted MCAO protocol. The rats were administered asialo erythropoietin at either 1.5 hour or 4 hours prior to reperfusion. Three doses were used for −1.5 hr: 0, 5, 50 ug/kg bw (6 rats per dose) and two for −4 hrs: 0 and 50 ug/kg bw (6 rats per group). Read out of volume of injury was determined by tetrazolium staining of brain sections performed 24 hours later in accordance with the protocol set forth in Brines et al. 2000 Proc Natl Acad Sci USA 97:10526-10531.

FIG. 5 is a graph demonstrating the volume of lesions resulting from the MCAO protocols. Pretreatment with asialo erythropoietin showed significant reductions in the lesion volume when administered at 1.5 hours and 4 hours prior to the MCAO surgery.

B. Therapeutic Window of Tissue Protective Cytokines

A MCAO protocol as outlined above was used for the present example. Following the procedure, PBS or carbamoylated erythropoietin (10 ug/kg, i.v.) were administered to the rats at 24 hours and 48 hours.

Subsequently, the rats were subjected to behavioral testing. The de Ryck test, set forth in De Ryck et al., Noecortical localization of tactile/proprioceptive limb placing reactions in the rat, Brain Research, 573 (1992) 44-60, was performed over a period of 28 days to assess the limb placement reaction in the treated rats. In accordance with the De Ryck test each of the rats was subjected to six tests. Four tests measured: how the rat stretches its legs by holding rat 10 cm above a table nose downwards; whether rat keeps in contact with the table by holding rat at the edge of the table and its head at a 45 degree angle; how the rat places the front paws on the table by holding rat at a border of a table and observing whether he places his paws on the table; and measure if the rat “falls” from the table by placing the rat at the edge of a table and seeing whether he lets his front paws fall off the table. The rats were scored as follows 0, no placing; 1, incomplete or delayed placing of paws, and 2, immediate and complete placing of paws. Two additional tests measured whether the rat goes sidewards on a table by placing the rat parallel to the table and determining whether it attempts to place either front or hind paw on the table and whether the rat falls sidewards from the table by placing rat parallel to an edge of the table and determining if it will let either its front or hind paws fall off table when pushed. The same scoring system was used for these tests but the front and hind paws were graded separately. The maximum score a rat could achieve is 16. In FIG. 6, it is clear that the rats treated with carbamoylated erythropoietin at 24 and 48 hours after reperfusion had better placement scores than the control group indicating carbamoylated erythropoietin's therapeutic effect even when administered 24 hours following the injury.

The rats were also tested in a foot fault behavioral protocol. Rats were tested on an elevated stainless steel grid floor 30 cm×30 cm with grid size of 30 mm according to the protocol of Markgraf et al. Brain Research 575:238-246 (1992). When placed on the grid, rat would move around and occasionally a foot is misplaced and falls through a grid opening (“foot fault”). The number of foot faults was measured for a 1 minute period. As can be seen in FIG. 7, the rats treated with carbamoylated erythropoietin 24 hours and 48 hours following reperfusion suffered from less foot faults than those treated with PBS.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

What is claimed is:

1. A method for protecting or maintaining the viability of a responsive mammalian cell, tissue or organ comprising administering to a mammal a pharmaceutical composition comprising an erythropoietin or a tissue protective cytokine wherein the pharmaceutical composition is administered at a time outside a therapeutic window recognized for currently approved therapeutics for the injury.

2. The method of claim 1, wherein the pharmaceutical composition is administered prior to the therapeutic window recognized for a currently approved therapeutic for the injury.

3. The method of claim 1, wherein the pharmaceutical composition is administered after the therapeutic window recognized for the currently approved therapeutic for the injury.

4. A method for protecting or maintaining the viability of a responsive mammalian cell, tissue or organ from an injury comprising administering a pharmaceutical composition comprised of an erythropoietin or a tissue protective cytokine wherein the pharmaceutical composition is administered at least at any two of the following times: (1) prior to a therapeutic window recognized for a currently approved therapeutic for the injury, (2) within the therapeutic window recognized for a currently approved therapeutic for the injury, or (3) after the therapeutic window recognized for the currently approved therapeutic for the injury.

5. A method for protecting or maintaining the viability of a responsive mammalian cell, tissue or organ from an injury comprising administering a pharmaceutical composition comprised of an erythropoietin or a tissue protective cytokine wherein the pharmaceutical composition is administered at a time after the therapeutic window recognized for currently approved therapeutics for the injury and at least one of the following times: (1) prior to a therapeutic window recognized for a currently approved therapeutic for the injury, or (2) within the therapeutic window recognized for a currently approved therapeutic for the injury.

6. The methods of claims 1, 4 or 5, wherein the mammalian cell, tissue or organ is spinal cord.

7. The method of claim 6, wherein the trauma or injury is spinal cord injury.

8. The method of claim 7, wherein the tissue protective cytokine is administered outside the therapeutic window of methylprednisone.

9. The methods of claims 1, 4 or 5, wherein the mammalian cell, tissue or organ is brain.

10. The method of claim 9, wherein the trauma or injury is stoke or brain trauma.

11. The method of claim 10, wherein the tissue protective cytokine is administered outside the therapeutic window of recombinant tissue plasminogen activator.

12. The methods of claims 1, 4 or 5, wherein the pharmaceutical composition is capable of a therapeutic effect when administered at about 8 hours to about 168 hours following the injury.

13. The method of claim 12, wherein the pharmaceutical composition is capable of a therapeutic effect when administered at about 12 hours to about 72 hours following the injury.

14. The methods of claims 1, 4, or 5, wherein the pharmaceutical composition is capable of a therapeutic effect when administered at about 1 to 24 hours prior to the injury.

15. The method of claim 14, wherein the pharmaceutical composition is capable of a therapeutic effect when administered at about 6 to 18 hours prior to the injury.

16. The methods of claims 1, 4 or 5, wherein the mammalian cells comprise neuronal, retinal, muscle, heart, lung, liver, kidney, small intestine, adrenal cortex, adrenal medulla, capillary, endothelial, testes, ovary, endometrial, or stem cells.

17. The use of claim 16, wherein the mammalian cells further comprise photoreceptor, ganglion, bipolar, horizontal, amacrine, Müller, myocardium, pace maker, sinoatrial node, sinus node, atrioventricular node, bundle of His, hepatocyte, stellate, Kupffer, mesangial, goblet, intestinal gland, enteral endocrine, glomerulosa, fasciculate, reticularis, chromaffin, pericyte, Leydig, Sertoli, sperm, Graffian follicles, primordial follicles, endometrial stroma, and endometrial cells.

18. The methods of claims 1, 4 or 5, wherein the trauma or injury is caused by the injury is caused by a seizure disorder, multiple sclerosis, stroke, hypotension, cardiac arrest, ischemia, myocardial infarction, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis, alcoholism, mood disorder, anxiety disorder, attention deficit disorder, autism, Creutzfeldt-Jakob disease, brain or spinal cord trauma or ischemia, heart-lung bypass, chronic heart failure, macular degeneration, diabetic neuropathy, diabetic retinopathy, glaucoma, retinal ischemia, or retinal trauma.

19. The methods of claims 1, 4 or 5, wherein the erythropoietin is selected from the group consisting of a chemically modified erythropoietin and a recombinant erythropoietin.

20. The methods of claims 1, 4 or 5, wherein the tissue protective cytokine lacks at least one activity selected from the group consisting of increasing hematocrit, vasoconstriction, hyperactivating platelets, pro-coagulant activity and increasing production of thrombocytes.

21. The method of claim 20, wherein the tissue protective cytokine is selected from the group consisting of a chemically modified erythropoietin and a recombinant erythropoietin.

22. The method of claim 21, wherein the tissue protective cytokine is a chemically modified erythropoietin is selected from the group consisting of
i. An erythropoietin having at least no sialic acid moieties;
ii. An erythropoietin having at least no N-linked or no O-linked carbohydrates;
iii. An erythropoietin having at least a reduced carbohydrate content by virtue of treatment of native erythropoietin with at least one glycosidase;
iv. An erythropoietin having at least one or more oxidized carbohydrates;
v. A chemically reduced erythropoietin having at least one or more oxidized carbohydrates;
vi. An erythropoietin having at least one or more modified arginine residues;
vii. An erythropoietin having at least one or more modified lysine residues;
viii. An erythropoietin having at least one modification of the N-terminal amino group of the erythropoietin molecule;
ix. An erythropoietin having at least a modified tyrosine residue;
x. An erythropoietin having at least a modified aspartic acid or glutamic acid residue;
xi. An erythropoietin having at a modified tryptophan residue;
xii. An erythropoietin having at least one amino acid removed;
xiii. An erythropoietin having at least one opening of at least one of the cystine linkages in the erythropoietin molecule; and
xiv. A truncated erythropoietin.

23. The method of claim 22, wherein the tissue protective cytokine is asialo erythropoietin.

24. The method of claim 23, wherein said asialo erythropoietin is human asialo erythropoietin.

25. The method of claim 22, wherein the tissue protective cytokine is an erythropoietin having at least one carbamoylated lysine residue.

26. The method of claim 21, wherein the tissue protective cytokine is a recombinant erythropoietin comprising an erythropoietin mutein having one or more altered amino acid residue between position 11 to 15 of SEQ ID NO:5 [SEQ ID NO:1], position 44 to 51 of SEQ ID NO 5 [SEQ ID NO:2], position 100-108 of SEQ ID NO 5 [SEQ ID NO:3], or position 146-151 of SEQ ID NO 5 [SEQ ID NO:4].
